(19) United States
(12) Patent Application Publication
(10) Pub. No.: US 2010/0094141 A1
(43) Pub. Date: Apr. 15, 2010

(54) JUGULAR VENOUS PRESSURE RULER

(76) Inventor: Amal Lesly Puswella, Seattle, WA (US)

Correspondence Address: BLACK LOWE & GRAHAM, PLLC, 701 FIFTH AVENUE, SUITE 4800, SEATTLE, WA, 98104, US

(21) Appl. No.: 12/251,263

(22) Filed: Oct. 14, 2008

(51) Int. Cl. A61B 5/024; A61B 5/021

(52) U.S. Cl. 600/485; 600/502

(57) ABSTRACT

A JVP ruler and a method for its use in measuring a jugular venous pressure in a patient, includes orienting the JVP ruler such that the second arm is collinear with a vertical line originating at a right atrium of the patient and such a the first arm is horizontal and having a transducer end situated opposite the pivot end of the first arm. The JVP Ruler has first and second arms elongate and situated to be in perpendicular relation one to the other. The arms meet and terminate at a pivot located at the pivot ends of the arms respectively, the transducer end being generally above a pulse point, the pulse point being a point on the skin of the patient where variations of the jugular venous pressure within the internal jugular vein are exhibited as at least vertical displacement of the skin.

### FIELD OF THE INVENTION

This invention relates generally to medical diagnostic tools and, more specifically, to cardiology diagnostic tools.

### BACKGROUND OF THE INVENTION

The jugular venous pressure (JVP) is the indirectly observed pressure over the venous system. As a convention herein, JVP relates to the instantaneous pressure in an internal jugular vein; “jugular venous pulse” refers to variations of the JVP over the period of one complete cycle of the beating of a heart. Knowing the JVP can be useful in the differentiation of different forms of heart and lung disease. Classically, three upward deflections and two downward deflections have been described. The upward deflections are the “a” (atrial contraction), “c” (ventricular contraction and resulting bulging of tricuspid into the right atrium during isovolumic systole) and “v”=atrial venous filling. The downward deflections of the wave are the “x” (the atrium relaxes and the tricuspid valve moves downward) and the “y” descent (filling of ventricle after tricuspid opening).

The anatomic relationships of the right internal and external jugular veins to the right atrium are important to an understanding of the clinical evaluation of the venous pulse. The right internal jugular vein communicates directly with the right atrium via the superior vena cava. There is a functional valve at the junction of the internal jugular vein and the superior vena cava. Usually, however, this valve does not impede the phasic flow of blood to the right atrium. Thus the wave form generated by phasic flow to the right atrium is accurately reflected in the internal jugular vein. The external jugular vein descends from the angle of the mandible to the middle of the clavicle at the posterior border of the sternocleidomastoid muscle. The external jugular vein possesses valves that are occasionally visible. The relatively direct line between the right external and internal jugular veins, as compared to the left external and internal jugular veins, make the right jugular vein the preferred system for assessing the venous pressure and pulse contour.

In determining mean jugular venous pressure, one assumes that the filling pressure of the right atrium and right ventricle mirror that of the left atrium and left ventricle. This relationship is usually correct. Thus, a mean jugular venous pressure greater than 10 cm H₂O usually indicates volume overload, while a low jugular venous pressure (i.e., less than 5 cm H₂O) usually indicates hypovolemia. But there are important, notable exceptions to this relationship. First, acute left ventricular failure (as may be caused by a myocardial infarction) may significantly raise the pulmonary capillary wedge pressure without raising the mean right atrial and jugular venous pressures. Second, pulmonary hypertension, tricuspid insufficiency, or stenosis may be associated with elevated mean right atrial and jugular venous pressures while leaving the left heart pressures unaffected. In using the mean jugular venous pressure in clinical practice, the physician must correlate this bedside measurement with the other information gained from the history and physical examination.

Certain wave form abnormalities, include “Cannon a-waves”, which result when the atrium contracts against a closed tricuspid valve, due to complete heart block (3rd degree heart block), or even in ventricular tachycardia. Another abnormality, “c-v waves”, can be a sign of tricuspid regurgitation.

An elevated JVP is the classic sign of venous hypertension (e.g. right-sided heart failure). JVP elevation can be visualized as jugular venous distension, whereby the JVP is visualized at a level of the neck that is higher than normal. The paradoxical increase of the JVP with inspiration (instead of the expected decrease) is referred to as Kussmaul's sign, and indicates impaired filling of the right ventricle. The differential diagnosis of Kussmaul's sign includes constrictive pericarditis, restrictive cardiomyopathy, pericardial effusion, and severe right-sided heart failure.

Referring to FIG. 1, the jugular venous pulsation has a double waveform. The “a” wave corresponds to atrial contraction and ends synchronously with the carotid artery pulse. The “c” wave occurs when the ventricles begin to contract and is caused by bulging of the atrioventricular (AV) valves backwards towards the atria. The “x” descent follows the “c” wave and represents atrial relaxation and rapid filling due to low pressure. The “v” wave is seen when the tricuspid valve is closed and is caused by a pressure increase in the atrium as the venous return fills the atria—with and just after the carotid pulse. The “y” descent represents the rapid emptying of the atrium into the ventricle following the opening of the tricuspid valve. The absence of “a” waves is a feature of atrial fibrillation. “Cannon a waves” or increased amplitude “a” waves, are associated with AV dissociation (third degree heart block), when the atrium is contracting against a closed tricuspid valve.

The JVP is generally observed if one looks along the surface of the sternocleidomastoid muscle, as it is easier to appreciate the movement relative the neck when looking from the side (as opposed to looking at the surface at a 90 degree angle). Like judging the movement of an automobile from a distance, it is easier to see the movement of an automobile when it is crossing one's path at 90 degrees (i.e. moving left to right or right to left), as opposed to coming toward one.

Pulses in the JVP are rather hard to observe, but trained cardiologists do try to discern these as signs of the state of the right atrium. Nonetheless, measurement of JVP tends to be subjective, varying from one observer to another. Additionally, the current method is not susceptible to comparison between a current and a prior measurement. As infirmities of the heart tend to be progressive, knowledge of progress of the disease as measured by means of regular examination, objective measurement of the JVP, and recording of the results of the examination could yield extremely useful data in assessment of the patient's state of health.

What is missing in the art is a method of recording an objective reproducible means of measurement of the JVP for inclusion in medical chart.

### SUMMARY OF THE INVENTION

A JVP ruler and a method for its use in measuring a jugular venous pressure in a patient includes orienting the JVP ruler such that the second arm is collinear with a vertical line originating at a right atrium of the patient and such that the first arm is horizontal and having a transducer end situated opposite the pivot end of the first arm. The JVP Ruler has first and second arms elongate and situated to be in perpendicular relation one to the other. The arms meet and terminate at a pivot located at the pivot ends of the arms respectively, the transducer end being generally above a pulse point, the pulse point being a point on the skin of the patient where variations of the jugular venous pressure within the internal jugular vein are exhibited as at least vertical displacement of the skin. The JVP ruler is translated along the vertical line such that a rest end opposite the pivot end of the second arm is resting on the sternal angle of the patient approximately 5 centimeters above the right atrium. The transducer, located generally at the transducer end, is aimed at the pulse point such that when the transducer is activated, it can detect the vertical displacement. The transducer is activated for a period sufficient to record signals indicative of at least the vertical displacement, over at least one period of a jugular venous pulse in a memory.

### BRIEF DESCRIPTION OF THE DRAWINGS

The preferred and alternative embodiments of the present invention are described in detail below with reference to the following drawings.

FIG. 1 is a pressure curve reflecting a single cycle of the jugular venous pulse;

FIG. 2 is an exterior view of a JVP ruler;

FIG. 3 is a cutaway view of the JVP ruler;

FIG. 4 is a view of the JVP ruler in use over a patient; and

FIG. 5 is a flow chart reflecting the method of use of the JVP ruler.

### DETAILED DESCRIPTION OF THE PREFERRED EMBODIMENT

By way of overview, a JVP ruler and a method for its use in measuring a jugular venous pressure in a patient, includes orienting the JVP ruler such that the second arm is collinear with a vertical line originating at a right atrium of the patient and such a the first arm is horizontal and having a transducer end situated opposite the pivot end of the first arm. The JVP Ruler has first and second arms elongate and situated to be in perpendicular relation one to the other. The arms meet and terminate at a pivot located at the pivot ends of the arms respectively, the transducer end being generally above a pulse point, the pulse point being a point on the skin of the patient where variations of the jugular venous pressure within the internal jugular vein are exhibited as at least vertical displacement of the skin. The JVP ruler is translated along the vertical line such that a rest end opposite the pivot end of the second arm is resting on the sternal angle of the patient approximately 5 centimeters above the right atrium. The transducer, located generally at the transducer end, is aimed at the pulse point such that when the transducer is activated, it can detect the vertical displacement. The transducer is activated for a period sufficient to record signals indicative of at least the vertical displacement, over at least one period of a jugular venous pulse in a memory. Jugular venous pulsation and jugular venous pressure means “internal” jugular venous pulsation and pressure. Although the internal jugular vein is deep to the sternocleidomastoid muscle, pulsation of the column of blood within the vein is visible beneath the skin. The pulsation does not arise from the vein but reflects changes in pressure within the right atrium. The right internal jugular vein provides a more direct channel from the right atrium than the left and inspection of the right internal jugular vein for pulsation and pressure is a better choice. Inspection of the external jugular vein for pulsation and pressure is a poor alternative to inspection of the internal jugular vein because the external vein has valves and passes through fascial planes. Both factors can mask the transmission of pulse and pressure from the right atrium.

Three positive waves (a, c, & v) and 2 negative descents (x & y) have been described for internal jugular venous pulsation, although the c wave is so small in a normal subject that it is usually not visible to the naked eye. The positive a wave is the most prominent; it represents right atrial contraction. As the right atrium contracts, venous blood is pumped across the triscupid valve into the right ventricle but backpressure is also transmitted to the valveless internal jugular vein.

The highest point of this pulsating column of blood is called the head. The height of this head varies somewhat with respiration: falls slightly with inspiration when the negative intra-thoracic pressure encourages venous return to the heart; rises again with expiration when the positive intra-thoracic pressure impedes venous return to the heart. The mean height of this column (averaged over inspiration and expiration) represents the hydrostatic pressure within the right atrium, the normal magnitude of which is 6-10 cm H₂O. Jugular venous pressure (JVP) is commonly expressed as the vertical height (in cm) of this column of blood (the head) in relation to the sternal angle (angle of Louis).

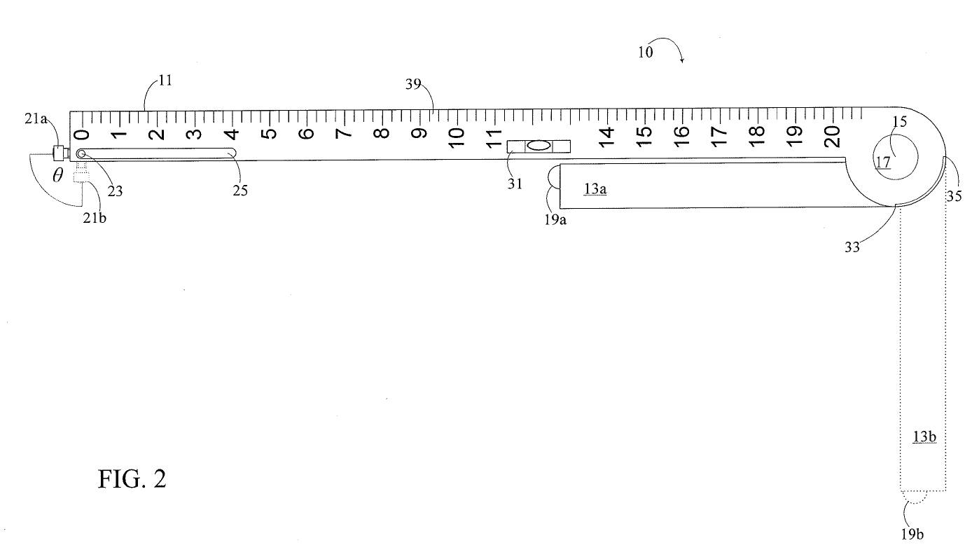

FIG. 2 depicts a JVP Ruler 10 having a first arm 11 including a pivot at a pivot 15 at a pivot end and a transducer 21a, 21b. A second arm 13a, 13b rotates about the pivot 15 from a parallel position 13a to a perpendicular position 13b (shown in phantom). A rest 19a, 19b is affixed to the second arm 13a, 13, in opposed relation to the pivot 15. The first arm 11 includes a stationary stop 35 as the second arm includes a rotating stop 33. The stops 33, 35 are arranged such that when the rotation of the second arm 13 about the pivot 15 brings a rotating stop 33 into contact with the stationary stop 35 the second arm 13 is held in perpendicular relation to the first arm 11.

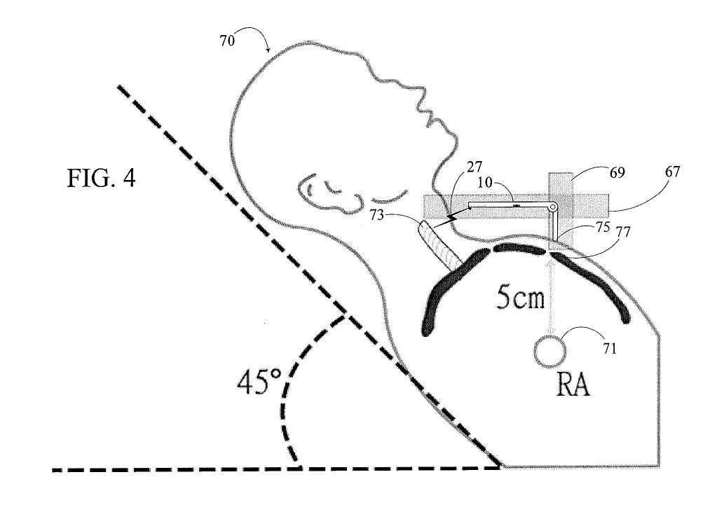

In an embodiment of the JVP ruler 10, a switch button 17 is located over the pivot 13. This optional and nonlimiting location of the switch button 17 allows a physician to grasp the JVP ruler 10 in a manner that allows the selective activation of internal circuitry while suitably holding the JVP ruler 10 in position over a patient as discussed in the context of FIG. 4 below.

An electronic transducer 21a, 21b (shown in phantom) is affixed to a pin 23 rotatably set in a channel 25. The transducer 21a, 21b can be rotated from a first position 21a through an angle θ to a position 21b, the pin 23 being slidingly engaged with the channel 25 in this nonlimiting embodiment. The transducer 21 can, by means of the pin 23 rotated and slid within the channel 25, be suitably aimed at a patient's skin situated immediately over the internal jugular vein. When the transducer 21 is suitably aimed, the transducer 21 can detect displacement of the patient's skin outwardly. Because, in the presently preferred embodiment, the angle θ can be determined, movement of skin can be resolved to determine a vertical displacement of the skin.

In the portrayed nonlimiting embodiment of the JVP Ruler 10, a bubble vial 31 is advantageously placed upon the first arm 11 to assist the physician in maintaining the first arm 11 in a horizontal position. In further alternate embodiments, the vials 31 are used in conjunction with an electro optical system which can accurately detect the bubble position within the vial 31. The position of the bubble can then be used to provide feedback to set the first arm 11 level or to determine an angle of displacement of the first arm 11 from the horizontal, which, when calculated with the angle θ and the known displacement of the pin 23 from the pivot 15, can facilitate the exact calculation of the position and angle of the transducer 21 relative to the patient's sternal angle (discussed in conjunction with FIG. 4 below).

In a preferred embodiment of the JVP Ruler, a camera is the transducer 21 such that a field of vision the camera entails defines a transducer cone. Movement of the skin within the transducer cone can be noted relative to the generally stationary surrounding skin. In an alternate embodiment, the transducer 21 is a sonar transducer such as those used in surgical robotic devices to locate a surface of a tissue. In still another embodiment, an array of IR emitters can also serve as the transducer 21 to measure the displacement of the skin immediately over the internal jugular vein. In still another embodiment, the transducer includes a plunger, the plunger being placed in contact with the skin immediately over the internal jugular vein thereby yielding displacement of the skin axially to the orientation of the plunger. Any transducer 21 configured to measure the displacement of the skin immediately over the internal jugular vein, will suitably serve the purposes of the JVP Ruler 10.

In any of the optical embodiments of the transducer 21, an added advantage exists. By way of non-limiting explanation, the camera embodiment will demonstrate the advantage. Where a bar coded identification exists, the JVP Ruler 10 will suitably read the bar code to obviate the need for manual entry of the results of the examination. By directing the transducer at the bar code, thereby eliminating the possibility of scrivener's errors in recording the patient's identity. While the same advantages can be achieved with a dedicated camera in an alternate embodiment, dual tasking of the transducer 21 is an advantage the optical transducer 21 embodiments afford.

A second opportunity for dual tasking is afforded by the inclusion, in an embodiment, of a scale 39. In at least one embodiment, a metric scale 39, is placed on the JVP Ruler 10 in order to assist a physician in examinations distinct from the primary use of the JVP ruler 10.

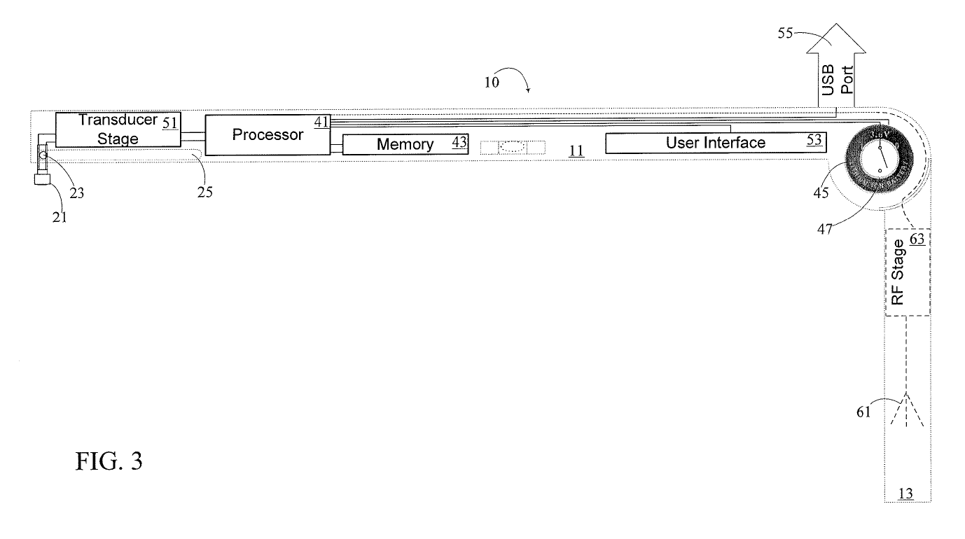

FIG. 3 depicts an embodiment of circuitry that will enable the JVP Ruler 10. A power supply 45 (in this nonlimiting embodiment, a lithium ion battery) provides energy to the supporting circuitry. In alternate embodiments, voltage regulation might be included and alternate means may exist for storing power. Indeed, because of the sporadic nature of heavy drain, kinetic power supplies 45 such as those used in watches such as the Kinetic™ line by Seiko™ may well serve. For nonlimiting purposes of explanation, a battery fulfills the role of power supply 45, though other known technologies exist.

The power supply 45 conducts current to the processor 41, which may include onboard random access memory RAM and various buffers. Additionally, memory 43 is provided. In the presently preferred embodiment, the memory 43 is flash memory available upon which will reside firmware as well as any persistent memory, such as might be necessary for recording the measurements by the transducer 21. As so configured, the processor 41 receives positional data (angle and placement on the JVP ruler 10) of the transducer and then converts transducer 21 measurements of displacement of the skin immediately covering the internal jugular vein. The processor resolves the displacement into x-axis and y-axis components, recording the x-axis displacement as indicative of jugular venous pressure. These results are stored in memory 43 and in some embodiments, associated with a patient identifier.

Also shown is a transducer stage 51 upon which all of the supporting and driving electronics are located to facilitate operative communication between the processor and the transducer. While in many embodiments, no such transducer stage 51 is necessary to facilitate that communication, for clarity of explanation of purpose, the transducer stage 51 is portrayed. In operation, when requested, by the processor 41, the transducer 21 measures and transmits the displacement vectors for the skin immediately over the internal jugular vein (vectors referring to both a magnitude and a direction). Based upon the displacement vectors, the processor 41 generates a dataset to represent at least one cycle of the jugular venous pressure. That cycle is stored in memory 43 for retrieval and, in some embodiments, display 44.

A switch 47 is provided in the nonlimiting exemplary JVP Ruler 10 to allow an examining physician to indicate to the JVP Ruler 10 when the JVP Ruler 10 is suitably positioned for the measurement. Additionally, two means are shown to facilitate the communication of data from the JVP Ruler 10—a data port 55 (shown here, by way of nonlimiting example as a USB port) and a Radio Frequency Stage 63 and Antenna 61. In this embodiment, the Radio Frequency Stage 63 and Antenna 61 may be, for example, an 802.11 device or a Bluetooth™ device.

The 802.11 family includes over-the-air modulation techniques that use the same basic protocol. The most popular are those defined by the 802.11b and 802.11g protocols, and are amendments to the original standard. 802.11a was the first wireless networking standard, but 802.11b was the first widely accepted one, followed by 802.11g and 802.11n. 802.11b and 802.11g use the 2.4 GHz ISM band, operating in the United States under Part 15 of the US Federal Communications Commission Rules and Regulations. Because of this choice of frequency band, 802.11b and 802.11g equipment may occasionally suffer interference from microwave ovens and cordless telephones. Bluetooth™ devices, while operating in the same band, in theory do not interfere with 802.11b or 802.11g because they use a frequency hopping spread spectrum signaling method (FHSS) while 802.11b or 802.11g use a direct sequence spread spectrum signaling method (DSSS). 802.11a uses the 5 GHz U-NII band, which offers 8 non-overlapping channels rather than the 3 offered in the 2.4 GHz ISM frequency band.

As configured with the Radio Frequency Stage 63 and Antenna 61, the JVP Ruler 10 is capable of communicating with an internal hospital wireless Local Area Network, and in that capacity would be capable of almost immediate inclusion of the test results in the relevant patient's medical chart.

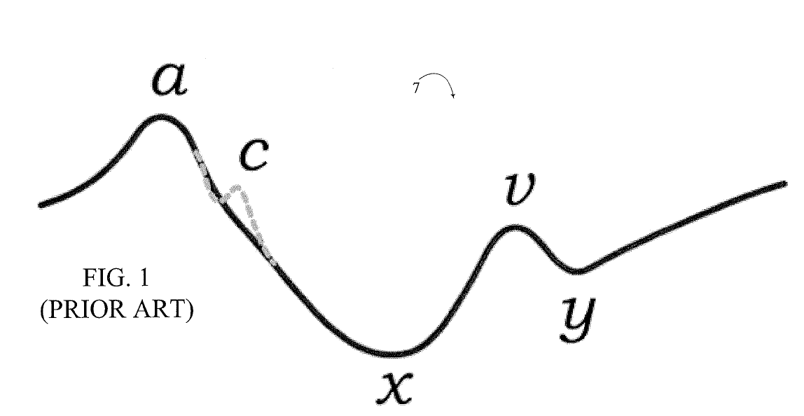

A User Interface 53 is optionally provided to allow the physician to have immediate feedback from the examination. A simple embodiment may include either of a lighted LED or audible tone to indicate a viable measurement. In more elaborate embodiments, a touch screen interface allows a physician to input patient data, shows the form of the curve upon measurement, presents a menu for selecting functions, and, after comparison with a stored series of typical measured curves, suggests to the physician possible conditions from which the patient may suffer, based upon noted aberrations from the idealized curve 7 (FIG. 1).

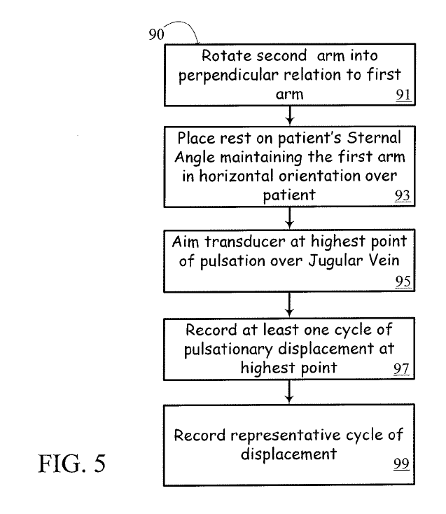

Referring to FIGS. 4 and 5, a method 90 for examination of a patient with a JVP Ruler 10 is set forth. Prefatory to the examination is the task of placing the patient in the suitable posture for testing. The patient 70 is shown in that position, such that the body assumes a 45° angle relative to the horizontal in what is, essentially, a reclining position. The patient 70 is well supported in the 45° position to avoid tensing of the sternocleidomastoid muscle. Should the patient 70 attempt to support themselves, thus tensing of the sternocleidomastoid muscle to support a poorly supported head, the tightened sternocleidomastoid muscle can prevent transmission of the internal jugular venous pulse to the skin.

At a block 91, the JVP Ruler 10 is opened by rotation of the second arm 13 (FIG. 2) such that the second arm 13 is at a right angle to the first arm 11 (FIG. 2). Once so opened, at a block 93, the JVP Ruler 10 is placed such that the second arm 13 is in a vertical plane 69 that the vertical plane 69 intersects the right atrium (RA). The rest 19 at the now lower end of the second arm 13 (opposite the pivot 15) rests approximately 5 cm above the RA.

Once so positioned, at a block 95, the transducer 21 (FIGS. 2, 3) is aimed at the patient 70, specifically at that part of the skin of the neck moved by the pulsating column of blood and its head in the right internal jugular vein 73. For ease of discussion, the area wherein the transducer 21 can determine the movement of skin shall be known as the transducer cone 27. For purposes of this convention, the transducer 21 is aimed at the pulsating internal jugular vein 73 such that the pulsating skin is within the transducer cone 27.

In the nonlimiting embodiment portrayed, the switch button 17 (FIG. 2) is depressed to begin the period of transducer 21 sensitivity. At a block 97, recording of the measurement begins. In this nonlimiting embodiment, at least one but generally a plurality of cycles of the jugular venous pressure are recorded. In a presently preferred embodiment, the physician initiates the scan and then the processor 41 continues to conduct the scan by the transducer 21 until there is little variation among several of the scans. At such a point, the most representative scan is selected as the measured scan and the transducer 21 returns to a state of nonsensitivity and some indication of completion is sent to the physician through the user interface 53.

While the preferred embodiment of the invention has been illustrated and described, as noted above, many changes can be made without departing from the spirit and scope of the invention. For example, the JVP Ruler might include a digital dictation capability that allows the physician to dictate medical chart notes during the exam and to send them for appropriate addition to the chart. Accordingly, the scope of the invention is not limited by the disclosure of the preferred embodiment. Instead, the invention should be determined entirely by reference to the claims that follow.

The embodiments of the invention in which an exclusive property or privilege is claimed are defined as follows:

1. A method for measuring a jugular venous pressure in a patient, the method comprising:
orienting a JVP Ruler, having first and second arms elongate and situated to be in perpendicular relation one to the other, meeting and terminating at a pivot at pivot ends respectively, such that the second arm is collinear with a vertical line originating at a right atrium of the patient and such that the first arm is horizontal and having a transducer end situated opposite the pivot end of the first arm, the transducer end being generally above a pulse point, the pulse point being a point on the skin of the patient where variations of the jugular venous pressure within the internal jugular vein are exhibited as at least vertical displacement of the skin;
translating the JVP ruler along the vertical line such that a rest end opposite the pivot end of the second arm is resting on the sternal angle of the patient approximately 5 centimeters above the right atrium;
aiming a transducer, located generally at the transducer end, at the pulse point such that when the transducer is activated, it can detect the vertical displacement;
activating the transducer for a period sufficient to record signals indicative of at least the vertical displacement, over at least one period of a jugular venous pulse in a memory.

2. The method of claim 1, wherein orienting is orienting with reference to a bubble vial the first arm includes.

3. The method of claim 1, wherein aiming includes rotating the transducer in the plane defined by the first and second arms.

4. The method of claim 1, wherein aiming includes
translating the transducer along the first arm.

5. The method of claim 1, wherein activating the transducer includes:
processing the signal from the transducer indicative of at least the vertical displacement to produce data indicative of variations of jugular venous pressure over at least one cycle of the jugular venous pulse.

6. The method of claim 5, wherein the processing includes storing the data indicative of variations of jugular venous pressure.

7. The method of claim 6, further including uploading the data indicative of variations of jugular venous pressure by means of one of a group consisting of an output port and a RF stage mated to an antenna.

8. The method of claim 7, wherein the RF stage mated to an antenna is according to the IEEE 802.11 family of protocols.

9. A jugular venous pulse (JVP) ruler for measuring variation in jugular venous pressure in the internal jugular vein of a patient, the JVP ruler comprising:
a pivot;
an elongate first arm having a first arm pivot end that terminates at a pivot and in opposed relationship to the first arm pivot end, a transducer end;
an elongate second arm having a second arm pivot end and rotatably joined to the first arm such that the second arm can rotate about the pivot from a position generally parallel to the first arm to a position generally perpendicular to the first arm, the first arm terminating in a rest end in opposed relation to the second arm pivot end; and
a transducer assembly, located generally at the transducer end and when the transducer assembly is situated generally above a pulse point, the pulse point being a point on the skin of the patient where variations of the jugular venous pressure within the internal jugular vein are exhibited as at least vertical displacement of the skin, at the pulse point such that when the transducer activated is activated, the transducer assembly can detect the vertical displacement.

10. The JVP ruler of claim 9 wherein the transducer assembly includes:
a power supply;
a transducer; and
a processor receiving power from the power supply and receiving a signal from the transducer representative of at least the vertical displacement and producing data representing jugular venous pressure as it varies through the period of a jugular venous pulse.

11. The JVP ruler of claim 10, wherein the processor includes a transducer stage facilitating operative communication between the transducer and the processor.

12. The JVP ruler of claim 10, wherein the processor includes a user interface.

13. The JVP ruler of claim 10, wherein the processor includes an output port for uploading data representing jugular venous pressure as it varies through the period of a jugular venous pulse.

14. The JVP ruler of claim 10, wherein the processor includes an RF stage and antenna for transmitting data representing jugular venous pressure as it varies through the period of a jugular venous pulse to a remote receiver.

15. The JVP ruler of claim 10, wherein the processor includes a memory for storing data representing jugular venous pressure as it varies through the period of a jugular venous pulse.

16. The JVP ruler of claim 9, wherein the second arm includes a rest generally at the rest end, the rest configured to contact the patient's sternal angle approximately 5 centimeters above the right atrium.

17. The JVP ruler of claim 9, wherein the first arm includes a scale for linear measurement.

18. The JVP ruler of claim 9, wherein the transducer assembly includes a pin to allow the transducer to rotate in the plane defined by the first and second arms.

19. The JVP ruler of claim 18, wherein the first arm defines a channel configured to allow the transducer to translationally move along the first arm.

20. The JVP ruler of claim 9, wherein the first arm includes a bubble vial to indicate an attitude of the first arm relative to the horizontal.
